(19) United States
(12) Patent Application Publication
(10) Pub. No.: US 2008/0300507 A1
(43) Pub. Date: Dec. 4, 2008

(54) Biopsy Needle

(75) Inventors: Stacy Figueredo, Arlington, MA (US); William J. Fienup, St. Louis, MO (US); Barry Kudrowitz, Cambridge, MA (US); Jacob A. Wronski, Mississauga (CA); Alexander H. Slocum, Bow, NH (US); William R. Brugge, Boston, MA (US)

Correspondence Address: WOLF GREENFIELD & SACKS, P.C., 600 ATLANTIC AVENUE, BOSTON, MA, 02210-2206, US

(73) Assignee: The General Hospital Corporation, Boston, MA (US)

(21) Appl. No.: 11/883,281

(22) PCT Filed: Jan. 30, 2006
(86) PCT No.: PCT/US06/03222

§ 371 Date: Apr. 16, 2008

Related U.S. Application Data
(60) Provisional application No. 60/648,175, filed on Jan. 28, 2005.

(51) Int. Cl. A61B 10/00

(52) U.S. Cl. 600/567

(57) ABSTRACT

A biopsy needle includes tissue capture elements within the needle lumen to help hold a tissue sample within the needle and maintain its integrity. According to one aspect, several flexible members form a structure that allows tissue to enter the lumen during advancement of the needle into tissue, and prevents the tissue sample from exiting the needle during retraction of the needle. The flexible members may, in some embodiments, include cutting edges configured to cut the tissue sample from the target tissue mass at the start of needle extraction.

### FIELD OF THE INVENTION

The invention relates generally to biopsy needles, and more particularly to methods and apparatus for collecting and retaining tissue samples in endoscopic biopsy needles.

### BACKGROUND OF THE INVENTION

Endoscopic biopsy is a minimally invasive medical procedure for detecting various types of cancer. During a biopsy, tissue samples are removed from the body and analyzed. Doctors use the biopsy samples to analyze the cellular composition of tissue, and in core-sampling biopsies, the histology (structure) of the tissue.

According to a national health study performed in 1996, over 1.2 million endoscopic biopsies are performed in the United States each year. Unfortunately, the success rate of obtaining a tissue sample with a coring biopsy needle is less than desired. In many cases, the obtained samples have poor cellular architecture quality and are not adequately intact to provide histological data.

Typical endoscopes include a hollow tube and a control handle. The hollow tube provides a conduit for safe insertion of the needle into the body and the control handle allows the doctor to bend the endoscope head. Typically, the endoscope head contains an ultrasound device, a camera, and liquid or air flushing capabilities. For a typical oral procedure, the endoscope is inserted into a patient's mouth and navigated to the biopsy site. After the endoscope is in place, a needle is passed through the hollow tube of the endoscope.

A typical endoscope and needle assembly, such as the one shown in FIG. 1, includes an endoscope control handle 10, a hollow tube 12, a needle control handle 14, a needle 16 having a lumen 18, a sheath 20, a removable stylet 22. The sheath protects the interior of the endoscope from needle damage. The stylet travels through the lumen of the needle and prevents a needle with an open end tip 24 from collecting tissue before the end tip reaches the target sample site. Once the needle reaches the target sample site, the stylet is removed through the proximal end of the needle. A control handle is used to control the extension and location of the needle, and to remove the stylet.

Biopsy needles can be separated into two general types: side-cutting needles and end-cutting needles. A side-cutting needle typically includes a sliding sheath which is moved past an opening along the side of the needle. The sheath or needle is configured to cut the tissue and force the tissue into the interior lumen of the needle. An end-cutting needle typically has a slanted tip and a cutting leading edge for puncturing and cutting the tissue. Once the needle has been advanced over a column of tissue, the intent is for the tissue column to remain in the needle lumen when the needle is retracted. Some needle assemblies include an aspiration needle which uses suction to help retain the tissue sample.

A need exists for biopsy needles that increase the success rate of obtaining a tissue sample and/or improve the quality of the samples obtained.

### SUMMARY OF THE INVENTION

According to certain aspects of the invention, a biopsy needle includes tissue capture elements within the needle lumen to help hold a tissue sample within the needle and maintain its integrity. According to one aspect, several flexible members form a sort of “tissue check-valve” that allows tissue to enter the lumen during advancement of the needle into tissue, and prevents the tissue sample from exiting the needle during retraction of the needle. The flexible members may, in some embodiments, include cutting edges configured to cut the tissue sample from the target tissue mass at the start of needle extraction.

According to one embodiment, a biopsy apparatus includes a needle having an opening to permit tissue entry and having a sidewall defining a lumen, and a tissue capture element protruding into the lumen, the tissue capture element being configured to permit tissue to move past the tissue capture element within the lumen, and further configured to prevent tissue from exiting the needle via the opening.

According to another embodiment, a needle includes a distal tip having an opening with a first cross-sectional area, the distal tip further having a cutting leading edge. The needle further includes a first longitudinal portion of the needle, proximal to the distal tip, having a first passage with a second cross-sectional area that is smaller than the first cross-sectional area, and a second longitudinal portion of the needle, proximal to the first longitudinal portion of the needle, having a second passage with a third cross-sectional area that is larger than the second cross-sectional area.

According to a further embodiment, a method of obtaining a tissue sample includes inserting a distal section of a needle having a lumen into a tissue mass to force a column of tissue into the lumen, a portion of the column of tissue passing through a tissue capture element disposed within the lumen. The method further includes retracting the needle to remove the column of tissue from the tissue mass, the tissue capture element providing resistance to movement of the tissue in the distal direction that is greater than any resistance provided to movement of the tissue in the proximal direction through the tissue capture element.

### BRIEF DESCRIPTION OF THE DRAWINGS

Other advantages, features, and uses of the invention will become apparent from the following detailed description of non-limiting embodiments of the invention when considered in conjunction with the accompanying drawings, which are schematic and which are not intended to be drawn to scale. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

FIG. 1 shows a typical prior art endoscope and needle assembly;

FIG. 2a is a perspective view of a needle including flexible members angled in the proximal direction according to one embodiment of the invention;

FIG. 2b is a side view of the embodiment shown in FIG. 2a;

FIG. 2c is a front view looking into the needle of the embodiment shown in FIGS. 2a and 2b;

FIG. 3 is a front view looking into a needle which includes one flexible member, according to an alternative embodiment of the invention;

FIG. 4a is a cross-sectional side view of an alternative embodiment of the invention comprising a ring with a passage;

FIG. 4b is a front view of the embodiment shown in FIG. 4a;

FIG. 5 is a cross-sectional side view of another alternative embodiment of the invention comprising an opening in the sidewall;

FIG. 6 is a cross-sectional side view of another alternative embodiment of the invention comprising a needle having internal barbs; and

FIG. 7 shows a side view of a removable needle tip according to one aspect of the invention.

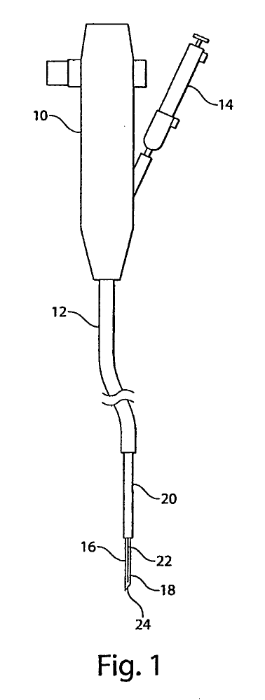

### DETAILED DESCRIPTION OF THE INVENTION

According to one aspect of the invention, methods and apparatus are provided to collect tissue samples by introducing tissue into a biopsy needle lumen during needle advancement and retaining the tissue sample in the needle lumen during needle retraction. According to another aspect of the invention, the amount of tearing that occurs during tissue sampling may be reduced by providing a cutting element within a biopsy needle. According to another aspect, the integrity of obtained samples may be improved, which can be particularly helpful when studying the histology of tissue samples. Not every embodiment of the invention includes all of the aspects of the invention described herein. Some embodiments do, however, include combinations of various aspects.

In some embodiments, the cutting and/or retaining of a tissue sample can be achieved in a passive manner, that is to say, active triggering of a cutting or retaining device by a user may not be required. Such an approach may reduce the complexity of use and/or the cost to manufacture.

Tissue capture elements, in some embodiments, are retaining members that protrude into the needle lumen and allow tissue to pass through a passage defined by the members and/or the interior wall of the needle. When the tissue contained within the needle is pulled to move in the opposite direction, i.e., during retraction of the needle from a tissue mass, the retaining members prevent the tissue from moving through the lumen by substantially constricting or closing off the previously available passage. In this manner, the retaining member or members act as a sort of one-way valve for the tissue sample.

In some embodiments, the retaining members do not open or constrict based on the movement of tissue through the lumen, but rather maintain a defined opening space or passage which acts as a one-way valve by virtue of the shape and structure of the members.

Instead of a plurality of members, in some embodiments a ring with a passage is used within a needle to provide a tissue capture element. The entrance to the passage, i.e., the side of the ring into which tissue is introduced, may be slanted so as to gradually compress the tissue as it moves through the ring. The passage may include a section of constant diameter, and then open relatively abruptly into the full lumen diameter of the needle on the exit side. The abrupt change in diameter (or cross-sectional area in the case of a non-cylindrical needle) prevents tissue from re-entering the passage on the exit side when the needle is retracted from the tissue mass and the sample of tissue tends to be pulled in such a direction. For purposes herein, the term “passage” means any opening, aperture, hole, pathway or channel through which tissues or cells may pass.

In embodiments where the retaining members move to expand and constrict a passage for the tissue, the members may be coupled to the needle sidewall with a flexural hinge. The members may extend from the needle wall at an angle and toward a proximal end of the needle. In other embodiments, other suitable hinges and/or attachments may be used to couple the members to the needle sidewall. For purposes herein, the proximal end of the needle apparatus is the end toward the control handle, while the distal end of the needle is the end which is introduced into a tissue mass.

The proximal side of the free ends of the retaining members may include a cutting surface such that upon needle extraction, when the tissue starts pushing against the back side of the needle members, the tissue is cut, thereby separating it from the main tissue mass. In this manner, the separation of the tissue sample from the tissue mass is passive. In other embodiments, active cutting devices may be used to separate the tissue sample. In still further embodiments, cutting of the tissue may be accomplished with a twisting action.

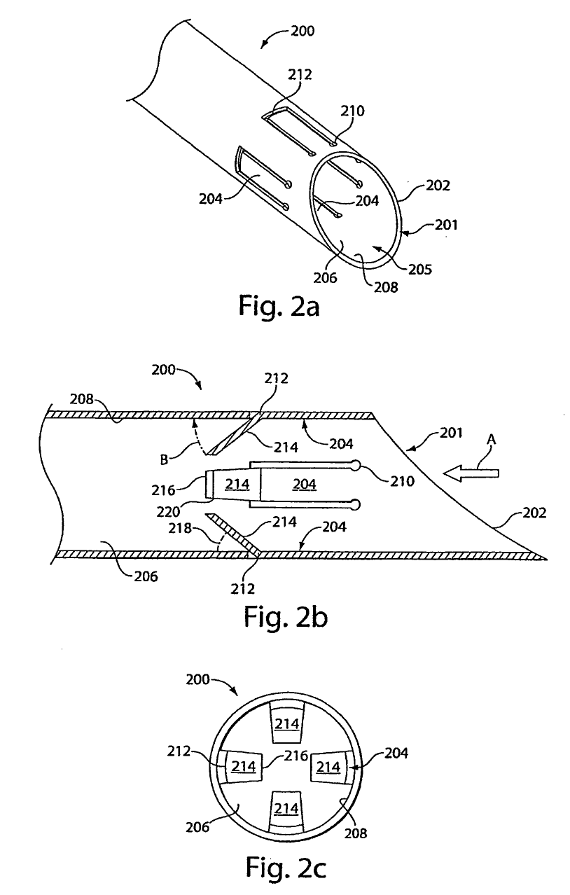

Referring now to the figures, one embodiment of a needle including a tissue capture element comprising four retaining members is shown in FIGS. 2a-2c. Needle 200 has a slanted distal tip 201 and includes a cutting leading edge 202 which circumferentially cuts tissue as needle 200 is advanced into a tissue mass (not shown). An opening 205 permits the introduction of tissue into a needle lumen 206. Four retaining members 204 extend from a needle sidewall 208 and are spaced symmetrically about a center axis of needle lumen 206.

Each retaining member 204 is coupled to needle 200 by a flexural hinge 210. To form this flexural hinge, a portion (or all) of retaining member 204 may be cut (such as by laser cutting), attached (such as by tacking or using adhesive), molded, or otherwise formed from needle wall 208. As shown in FIGS. 2a-2c, a bend 212 in the retaining member may be positioned at a distance from flexural hinge 210. Bend 212 provides retaining member 204 with a distally-facing surface 214. When tissue moves into needle 200 (i.e., in the direction of arrow A), the tissue pushes on distally-facing surface 214 and this force pushes retaining member 204 outwardly toward needle sidewall 208, i.e., in the direction of arrow B. This radial component of movement expands, or in some cases creates, a passage between the retaining members. The passage permits the further advancement of needle 200 into the tissue mass to push a column of tissue into lumen 206 and past tissue retaining members 204.

When a suitable amount of tissue has been introduced into needle 200 and pushed past retaining members 204, the needle is retracted. With some prior art needles, retraction of the needle results in the tissue column moving in a direction opposite to arrow A, i.e., out of needle 200, because the tissue column remains attached at its end to the tissue mass. In some embodiments of the present invention, as the tissue starts to move in the direction opposite to arrow A, sharp edges 216 of retaining members 204 catch the outer edge of the tissue column and further movement of the tissue column urges retaining members 204 radially inwardly which cuts the tissue. Additionally, the radially inward movement of retaining members 204 constricts, or in some cases closes, the passage that was previously present between the retaining members. In this manner, the tissue present in needle 200 is prevented from exiting via end opening 205. For purposes herein, urging a tissue capture element to constrict does not necessarily require the retaining member to completely close the lumen or any passage through which the tissue passed. Urging a tissue capture member to constrict encompasses (but is not limited to) urging the tissue capture element to constrict by an amount that does not represent the fullest possible constriction. Similarly, for purposes herein, urging a tissue capture member to open does not necessarily require the tissue capture member to completely open.

For purposes herein, lumen 206 is defined as extending to the distal end of the needle. As such, a retaining element that extends from the sidewall into an area partially radially bordered by cutting leading edge 202 is considered to be protruding into the lumen.

Bend 212 of retaining member 204 preferably forms an angle 218 with needle sidewall 208 of approximately 45°. In some embodiments, angle 218 may be between 30° and 60° inclusive, or another suitable angle. In some embodiments, retaining member 204 may not include a bend at all. For example, retaining member 204 could be coupled to the interior sidewall at an angle and not include a further bend. Or, in other embodiments, retaining member 204 may be parallel with sidewall 208 (or disposed at a very slight angle) and include a free end edge that is configured to grab tissue moving in a distal direction such that the retaining member 204 is pulled inwardly as the tissue movement continues.

While bend 212 is shown forming an acute angle with sidewall 208, retaining member 204 may form a 90° or an obtuse angle with the sidewall.

Retaining members 204 need not be longitudinally linear, but may include curves—convex or concave or both. In some embodiments, retaining member 204 may have a radius of curvature (relative to a longitudinal axis) identical to or similar to needle sidewall 208. In other embodiments, retaining members 204 may be flat in a side-to-side and/or longitudinal direction. In some embodiments, lateral sides 220 of retaining member 204 near sharp edge 216 may have a cutting edge. In this manner, twisting the needle may provide further cutting characteristics.

Four retaining members 204 are shown in the embodiment of FIGS. 2a-2c, but a fewer or a greater number of retaining members 204 may be used such as 1, 2, 3, 5, or 6 or more. Using an even number of retaining members 204 may provide advantages in manufacturability because if laser cutting is employed, opposing retaining members may be cut with a single cut. Stainless steel, or other suitable material, may be used to construct the needle and/or the retaining members. In some embodiments, the shapes and/or sizes of the retaining members may vary within the same needle 200. The longitudinal positioning of the retaining members may vary relative to one another and/or relative to cutting leading edge 202. Additionally, retaining members 204 need not be positioned radially symmetrically about the longitudinal axis of lumen 206.

The ends and/or sides of retaining members 204 may be constructed and arranged such that when they bend or move radially inwardly the ends and/or sides contact one another along complementary edges or surfaces. By doing so, a portion of a passage may be entirely blocked. For example, instead of having a flat distally-facing surface 214, retaining members 204 may be shaped such that when the four retaining members 204 are pushed together, there is no passage along the center axis of lumen 206 (although small passages may still exist radially outwardly from the center axis). In still other embodiments, retaining members 204 may be configured such that lumen 206 is entirely blocked when retaining members 204 are pushed together.

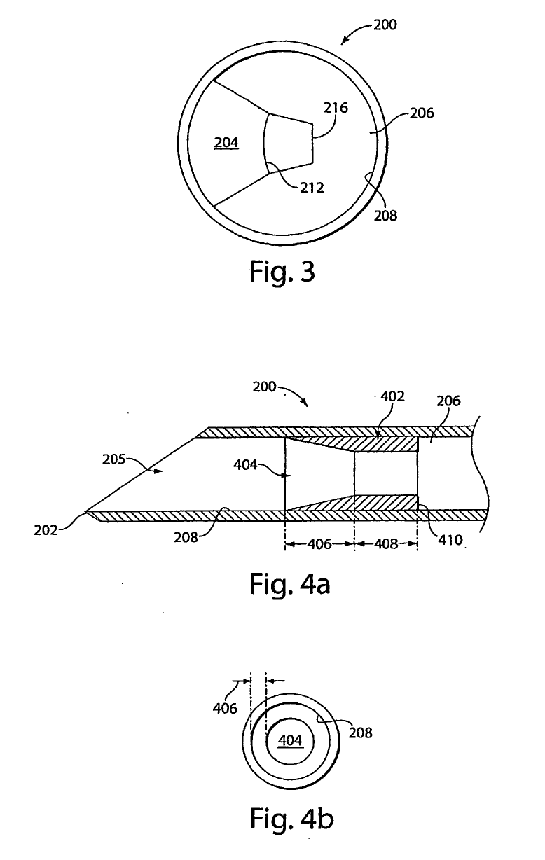

FIG. 3 shows an embodiment that includes a single retaining member 204 as a tissue capture element. This embodiment is similar in many respects to the embodiment shown in FIGS. 2a-2c, but instead of four retaining members, one large retaining member 204 is used to retain a tissue sample in needle 200. Retaining member 204 is shown with bend 212, although, as with the embodiment of FIGS. 2a-2c, a bend is not required. Retaining member 204 is coupled to sidewall 208 with a flexural hinge (not shown). For purposes herein, when describing one element as being “coupled to” another element, the term “coupled to” means any form of attachment (direct or indirect) and/or the elements being integral to one another. For example, in the embodiment of FIG. 3, retaining member 204 is coupled to sidewall 208 because retaining member 204 includes a portion that is cut from sidewall 208. Retaining member 204 would also be considered to be coupled to sidewall 208 if retaining member 204 is an independently manufactured element that is attached to sidewall 208 via adhesion or welding or other process.

As shown in this embodiment, a tissue capture element need not completely obstruct lumen 206 to prevent a tissue sample from exiting needle 200.

A stationary tissue capture element is shown in FIGS. 4a and 4b. In this embodiment, a ring 402 has a passage 404 which includes a longitudinal section that has a smaller cross-sectional area than lumen 206 of needle 200. Passage 404 is configured such that tissue can more easily travel proximally through passage 404 than the tissue can travel through passage 404 in the distal direction to exit the needle.

In the illustrated embodiment, ring 402 forms a passage that gradually narrows along a constricting length 406 in the proximal direction. A length 408 of constant diameter is present proximal to the constriction length 406. Proceeding proximally (to the right in FIG. 4a) passage 404 then opens into the full lumen diameter (or into a lumen having a diameter greater than the diameter of constant diameter section 408). With this configuration, tissue is cut into a column by leading edge 202, travels through distal end opening 205, is gradually compressed by constricting length 406, passes through constant diameter length 408, and passes into lumen 206 proximal to ring 402. When needle 200 is retracted from the tissue mass, a proximally-facing wall 410 of ring 402 prevents the collected tissue from exiting lumen 206 via passage 404. Constant diameter length 408 is not required, and passage 404 may proceed immediately from a constriction length 406 to opening to a larger lumen diameter.

Ring 402 may include sharp edges or cutting elements (not shown) on proximally-facing wall 410 to cut the connection of the tissue sample to the tissue mass. In some embodiments, proximally-facing wall 410 may have a slope instead of an abrupt diameter change, with the slope being steep enough to resist movement of tissue into passage 404 in the distal direction.

A stationary tissue capture element need not extend around the entire circumference of lumen 206. In some embodiments, non-flexible members similar in shape to the flexible retaining members illustrated in FIGS. 2a-2c may be employed. Instead of being thin members, however, the stationary members may be more volumetric, that is, similar to the ring in that they would have a larger contact area with sidewall 208 and have a proximally-facing wall similar to wall 410.

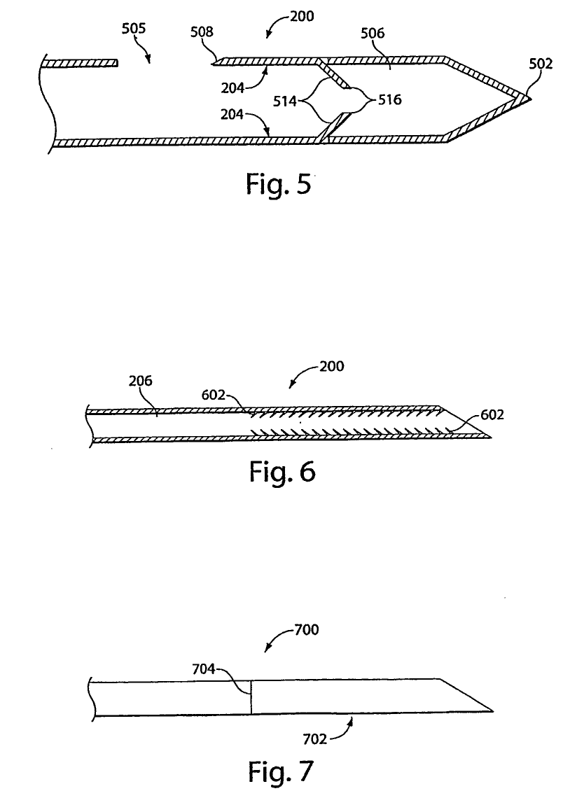

An alternative embodiment of the invention is shown in FIG. 5 in which a sidewall opening is used to introduce tissue into a needle rather than a distal end opening. In this embodiment, needle 200 is advanced into a tissue mass with a cutting leading edge 502. Tissue enters a lumen 506 through a sidewall opening 505. As needle 200 is retracted from the tissue mass, a cutting edge 508 cuts a length of tissue. Further retraction moves the tissue through retaining members 204. Force on a proximally-facing surface 514 enlarges a passage through lumen 506. As with the embodiment illustrated in FIGS. 2a-2c, retaining members 504 may include cutting elements 516, in this case facing in the distal direction. After tissue has moved past retaining members 504, movement of needle 200 in the distal direction may cause cutting elements 516 to shear the tissue from the tissue mass. In some embodiments, instead of, or in addition to, cutting edge 508, a sheath (not shown) that has a cutting edge may be used to partially or fully separate a tissue sample from the tissue mass. In some embodiments, the tissue capture element may be positioned proximal to sidewall opening 505 such that tissue passes the tissue capture element while needle 200 is being advanced.

While much of the description contained herein for various embodiments of the invention uses terminology associated with cylindrical devices (e.g., circumference, column, diameter), it is important to note that many of the embodiments may be employed using non-cylindrical components. For example, a needle having a square lumen may be used, or, in some embodiments, a passage in a ring or a passage through flexible retaining members may have a shape other than circular, cylindrical or substantially circular or cylindrical.

FIG. 6 shows an embodiment of a needle 200 in which a tissue capture element includes a plurality of angled barbs 602 disposed along the interior of needle sidewall 208. The cross-sectional view of FIG. 6 only shows sets of barbs protruding into the top and bottom of lumen 206, but similar sets of barbs 602 protrude into lumen 206 along the left and right sides of lumen 206 (as viewed from the distal end of the needle) as well. In this embodiment, twenty barbs are used per linear set of barbs, but other amounts may be used. Barbs 602 are angled in the proximal direction and may be any suitable thickness, such as approximately 0.1 mm. In some embodiments, fewer or greater numbers of sets of barbs or barbs per set may be used. The barbs illustrated in FIG. 6 are symmetrically disposed about a central axis of lumen 206, but in some embodiments, the barbs may be positioned asymmetrically. Additionally, barbs 602 need not be positioned linearly along needle 200. The angles that the barbs form with sidewall 208 may vary among the barbs. For example, in some embodiments, the barbs closer to the opening in the needle may form larger angles than the barbs that are farther from the opening.

In an alternative embodiment, the interior surface of needle 200 may be etched such that the coefficient of friction encountered by tissue differs depending on the direction that the tissue is moving or attempting to move. For example, the tissue capture element may include an etched surface in which tissue can more easily move into the needle as compared to the tissue moving toward the opening. In some embodiments, the etching feature may be combined with other tissue capture elements disclosed herein.

FIG. 7 illustrates an embodiment of a needle 700 in which a removable needle tip 702 is provided. With removable needle tip 702, a tissue sample may be removed from the needle assembly or endoscope assembly without removing the sample from the section of the needle in which the sample was originally collected. In some embodiments, this section of the needle may be made of a clear material (transparent or translucent) such that a doctor can visually confirm the presence of a sample without removing the sample from the needle. In some embodiments, the removable needle tip 702 may include a tissue capture element.

The removable needle tip may be made of polycarbonate, another plastic, or other suitable material, and removable using a shearing device or a scoring device at a selected longitudinal location 704. In some embodiments, the removable needle tip may be attached to a main needle body with threads and/or adhesive.

Removable needle tip 702 may be identified with an identifier such as a UPC symbol or an RFID tag before or after tissue collection to improve tracking of the tissue sample.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

We claim:

1. A biopsy apparatus comprising:
a needle having an opening to permit tissue entry and having a sidewall defining a lumen; and
a tissue capture element protruding into the lumen, the tissue capture element being configured to permit tissue to move past the tissue capture element within the lumen, and further configured to prevent tissue from exiting the needle via the opening.

2. A biopsy apparatus as in claim 1, wherein the opening in the needle is at a distal end of the needle.

3. A biopsy apparatus as in claim 2, wherein the distal end of the needle has a cutting edge along a border of the opening.

4. A biopsy apparatus as in claim 3, wherein the distal end of the needle has a slanted leading edge.

5. A biopsy apparatus as in claim 1, further comprising a retractable sheath, wherein the needle is positioned in the sheath.

6. A biopsy apparatus as in claim 1, wherein the tissue capture element is a flexible member which is coupled to the sidewall of the lumen.

7. A biopsy apparatus as in claim 1, wherein the tissue capture element comprises a plurality of members protruding into the lumen.

8. A biopsy apparatus as in claim 7, wherein the plurality of members comprise flexible members that form a passage, and which are bendable to change in a cross-sectional area of the passage.

9. A biopsy apparatus as in claim 7, wherein the plurality of members comprise flexible members which are bendable in a radial direction.

10. A biopsy apparatus as in claim 9, wherein the flexible members are constructed and arranged to bend inwardly and contact at least one other flexible member.

11. A biopsy apparatus as in claim 9, wherein at least one of the flexible members is constructed and arranged to bend inwardly and contact at least one other flexible member; and the contact between the two flexible members is by complementary edges or surfaces.

12. A biopsy apparatus as in claim 7, wherein the biopsy apparatus comprises between three and six members inclusive.

13. A biopsy apparatus as in claim 12, wherein the biopsy apparatus comprises four members.

14. A biopsy apparatus as in claim 7, wherein the members are arranged radially symmetrically about a central axis of the lumen.

15. A biopsy apparatus as in claim 7, wherein the members are disposed at approximately the same longitudinal position along the length of the needle.

16. A biopsy apparatus as in claim 7, wherein at least one of the members includes a cutting element.

17. A biopsy apparatus as in claim 1, wherein the tissue capture element is constructed and arranged such that movement of the tissue in the lumen toward the opening results in the tissue contacting the tissue capture element and urging the tissue capture element to close.

18. A biopsy apparatus as in claim 17, wherein the tissue capture element is constructed and arranged such that movement of the tissue in the lumen away from the opening results in the tissue contacting the tissue capture element and urging the tissue capture element to open.

19. A biopsy apparatus as in claim 17, wherein the tissue capture element is constructed and arranged such that movement of the tissue in the lumen toward the opening results in the tissue contacting the tissue capture element and closing the tissue capture element.

20. A biopsy apparatus as in claim 1, wherein the tissue capture element is constructed and arranged such that urging of the tissue toward the opening results in the tissue contacting and moving the tissue capture element such that a passage formed by the tissue capture element is smaller than when the tissue capture element is not contacted by tissue being urged toward the opening.

21. A biopsy apparatus as in claim 20, wherein the tissue capture element is constructed and arranged such that movement of the tissue away from the opening results in the tissue moving the tissue capture element such that the passage formed by the tissue capture element is larger than when the tissue capture element is not contacted by tissue moving away from the opening.

22. A biopsy apparatus as in claim 1, wherein, in a relaxed state, the tissue capture element is a member which is angled away from the opening and forms an angle with the sidewall of between 30 and 60 degrees inclusive.

23. A biopsy apparatus as in claim 22, wherein, in a relaxed state, the tissue capture element forms an angle with the sidewall of approximately 45 degrees.

24. A biopsy apparatus as in claim 1, wherein the tissue capture element is a flexible member having a thickness of approximately 0.1 mm.

25. A biopsy apparatus as in claim 1, wherein the needle comprises a plastic needle tip.

26. A biopsy apparatus as in claim 1, wherein the needle comprises a polycarbonate needle tip.

27. A biopsy apparatus as in claim 6, wherein the flexible member is integral to the sidewall.

28. A biopsy apparatus as in claim 27, wherein the flexible member is integrally attached to the sidewall by a flexural hinge.

29. A biopsy apparatus as in claim 27, wherein the flexible element is formed by laser cutting the sidewall.

30. A biopsy apparatus as in claim 1, wherein the tissue capture element is an element which is manufactured independently from the needle and attached to the needle sidewall.

31. A biopsy apparatus as in claim 1, wherein the tissue capture element is a ring with an interior passage that has a section with a smaller cross-sectional area than the lumen.

32. A biopsy apparatus as in claim 31, wherein the interior passage comprises a section which gradually decreases in cross-sectional area in a direction away from the opening.

33. A biopsy apparatus as in claim 32, wherein, further from the opening than the passage section which gradually decreases in cross-sectional area, the ring comprises an abrupt increase in cross-sectional area of the passage in a direction away from the opening.

34. A biopsy apparatus as in claim 33, wherein the abrupt increase in cross-sectional area occurs immediately adjacent to the smallest cross-sectional area of the interior aperture.

35. A biopsy apparatus as in claim 33, wherein the passage comprises a longitudinal section of substantially constant cross-sectional area which extends between the section that gradually decreases in area and the abrupt increase in area.

36. A biopsy apparatus as in claim 2, wherein the tissue capture element is configured to permit tissue to move past the tissue capture element within the lumen in the proximal direction, and further configured to prevent tissue from moving in the distal direction to exit the needle via the opening.

37. A biopsy apparatus as in claim 1, wherein the opening in the needle is within the sidewall.

38. A biopsy apparatus as in claim 37, wherein the tissue capture element is proximal to the opening in the sidewall.

39. A biopsy apparatus as in claim 37, wherein the tissue capture element is distal to the opening in the sidewall.

40. A biopsy apparatus as in claim 1, wherein the tissue capture element comprises barbs positioned at an angle to the sidewall.

41. An endoscope comprising the biopsy apparatus as recited in claim 1.

42. A needle, comprising:
a distal tip having an opening with a first cross-sectional area, the distal tip further having a cutting leading edge;
a first longitudinal portion of the needle, proximal to the distal tip, having a first passage with a second cross-sectional area that is smaller than the first cross-sectional area; and
a second longitudinal portion of the needle, proximal to the first longitudinal portion of the needle, having a second passage with a third cross-sectional area that is larger than the second cross-sectional area.

43. A needle as in claim 42, wherein the distal tip comprises a slanted leading edge.

44. A needle as in claim 42, wherein the needle has a cylindrical exterior.

45. A needle as in claim 42, wherein the first interior aperture is formed in an insert that is attached to an interior sidewall of the needle.

46. A needle as in claim 42, wherein the first interior aperture is formed by an interior sidewall of the needle.

47. A needle as in claim 42, wherein a transition from the first cross-sectional area to the second cross-sectional area is slanted.

48. A needle as in claim 42, wherein a transition from the second cross-sectional area to the third cross-sectional area is slanted.

49. A method of obtaining a tissue sample, comprising:
inserting a distal section of a needle having a lumen into a tissue mass to force tissue into the lumen, a portion of the column of tissue passing through a tissue capture element disposed within the lumen;
retracting the needle to remove a column of tissue from the tissue mass, the tissue capture element providing resistance to movement of the tissue in the distal direction that is greater than any resistance provided to movement of the tissue in the proximal direction through the tissue capture element.

50. A method of obtaining a tissue sample comprising:
providing a biopsy apparatus comprising:
a needle having an opening to permit tissue entry and having a sidewall defining a lumen; and
a tissue capture element protruding into the lumen, the tissue capture element being configured to permit tissue to move past the tissue capture element within the lumen, and further configured to prevent tissue from exiting the needle via the opening;
inserting the biopsy apparatus into a tissue mass; and
retracting the biopsy apparatus from the tissue mass.

51. A method of obtaining a tissue sample comprising:
providing a needle, comprising:
a distal tip having an opening with a first cross-sectional area, the distal tip further having a cutting leading edge;
a first longitudinal portion of the needle, proximal to the distal tip, having a first passage with a second cross-sectional area that is smaller than the first cross-sectional area; and
a second longitudinal portion of the needle, proximal to the first longitudinal portion of the needle, having a second passage with a third cross-sectional area that is larger than the second cross-sectional area;
inserting the needle into a tissue mass; and
retracting the needle from the tissue mass.
